(19) United States
(12) Patent Application Publication
(10) Pub. No.: US 2024/0317822 A1
(43) Pub. Date: Sep. 26, 2024

(54) PREPARATION OF ISOELECTRICALLY PRECIPITATED SUNFLOWER PROTEIN PRODUCTS WITH IMPROVED COLOUR

(71) Applicant: BURCON NUTRASCIENCE (MB) CORP., Winnipeg (CA)

(72) Inventors: Martin SCHWEIZER, Winnipeg (CA); Randy WILLARDSEN, Roseville, CA (US)

(73) Assignee: BURCON NUTRASCIENCE (MB) CORP., Winnipeg (CA)

(21) Appl. No.: 18/189,530

(22) Filed: Mar. 24, 2023

(51) Int. Cl. C07K 14/415; A23J 1/00; A23L 2/66; A23L 33/185; C07K 1/36

(52) U.S. Cl. CPC C07K 14/415; A23J 1/006; A23L 2/66; A23L 33/185; C07K 1/36

(57) ABSTRACT

A process for preparing a first sunflower protein product from a sunflower protein source, the sunflower protein product having a protein content of greater than 60, 65, 70, 75, 80, 85 or 90 wt % (N×6.25) d.b is provided. A food or beverage product comprising the sunflower protein product is also provided as is a sunflower protein product prepared using isoelectric precipitation.

### FIELD OF THE INVENTION

The present invention relates to sunflower protein products and methods of preparing sunflower protein products.

### SUMMARY OF THE INVENTION

The present invention relates to sunflower products, and processes for the preparation thereof. The sunflower protein product is prepared by isoelectric precipitation, with the extraction conditions such that the product has an improved colour.

In one embodiment, the present invention provides for a process for preparing a first sunflower protein product from a sunflower protein source, the sunflower protein product having a protein content of greater than 60, 65, 70, 75, 80, 85 or 90 wt % (N×6.25) d.b, the process comprising
a) extracting sunflower protein from an optionally partially defatted or optionally fully defatted, dehulled sunflower protein source, using an extraction solution comprising water containing an alkaline pH adjusting agent as well as ascorbic acid or a derivative thereof, to cause solubilization of the sunflower protein from the sunflower protein source to produce an aqueous phase and a residual sunflower protein source;
b) separating the aqueous phase from the residual sunflower protein source to produce an aqueous sunflower protein solution and a separated residual sunflower protein source;
c) optionally defatting the separated aqueous sunflower protein solution to at least partially remove fat from the separated aqueous sunflower protein solution;
d) adjusting the pH of the separated aqueous sunflower protein solution to an acidic value in the range of about pH 4.0 to about pH 5.0, preferably about pH 4.2 to about pH 4.8, to result in isoelectric precipitation of the sunflower protein;
e) separating the isoelectric precipitate from the remainder of the aqueous sunflower protein solution, termed the centrate;
f) optionally washing the isoelectric precipitate by mixing it with water with the pH of the system in the range of about 4.0 to about 5.0, preferably about pH 4.2 to about pH 4.8, and then separating the washed precipitate from the wash centrate;
g) optionally adjusting the pH of the optionally washed isoelectric precipitate to the range of about 6.0 to about 8.0, optionally about 6.5 to about 7.5, and optionally the isoelectric precipitate is in a form suited for use in neutral or near-neutral food applications;
h) optionally drying the optionally washed and optionally pH adjusted isoelectric precipitate;
wherein step a) is optionally carried out using a counter-current extraction procedure;
wherein the extraction is optionally carried out in a continuous operation or a batch operation; and/or
wherein step g) is optionally not performed and the isoelectric precipitate is in a form suited for use in low pH food and beverage applications.

In a further embodiment of the process or processes outlined above, the centrate formed in step e) may be combined with the wash centrate formed in step f) and concentrated and optionally diafiltered using membrane processing to provide a second sunflower protein product.

In a further embodiment of the process or processes outlined above, the dehulled sunflower protein source in a) is derived from confectionery (also known as non-oilseed), black oil (also known as oilseed) or conoil type sunflower seed.

In a further embodiment of the process or processes outlined above, the extraction solution contains ascorbic acid or a derivative thereof in an amount of from about 0.01 to about 1 wt % of the solution, preferably about 0.05 to about 0.15 wt %, more preferably about 0.05 to about 0.10 wt %.

In a further embodiment of the process or processes outlined above, the pH of the extraction is adjusted up to any value between the natural pH and about 8.5, or the pH may be adjusted within the range of about 7.0 to about 8.0, or the pH may be adjusted within the range of about 7.0 to about 7.5, wherein the alkaline pH adjusting agent in the extraction solution is food grade sodium hydroxide, food grade potassium hydroxide or any other conventional food grade alkali and combinations thereof.

In a further embodiment of the process or processes outlined above, solubilization of the protein in step a) is effected at a temperature of from about 1° to about 100° C., preferably about 15° to about 65° C., more preferably about 50° C. to about 60° C., preferably accompanied by agitation, for a time of about 1 to about 60 minutes, preferably about 10 to about 30 minutes.

In a further embodiment of the process or processes outlined above, the concentration of the sunflower protein source in the extraction solution during the extraction step is selected from the group consisting of about 5 to about 20% w/v and about 5 to about 15% w/v.

In a further embodiment of the process or processes outlined above, the aqueous phase resulting from the extraction step generally has a protein concentration of about 0.5 to about 5 wt %, preferably about 1 to about 5 wt %.

In a further embodiment of the process or processes outlined above, separation in step (b) comprises centrifugation and/or filtration optionally with a decanter centrifuge and a disc stack centrifuge and is conducted at the same temperature as the extraction step or at any temperature within the range of about 1° to about 100° C., preferably about 15° to about 65° C., more preferably about 50° to about 60° C.

In a further embodiment of the process or processes outlined above, the process further comprises treating the aqueous sunflower protein solution with an anti-foamer, such as any suitable food-grade, non-silicone based anti-foamer, to reduce the volume of foam formed upon further processing, wherein the quantity of anti-foamer employed is generally greater than about 0.0003% w/v, and wherein the anti-foamer is optionally added during the extraction step a).

In a further embodiment of the process or processes outlined above, defatting of the separated aqueous sunflower protein solution in step c) is achieved by centrifugation and/or filtration and/or the use of a three-phase centrifuge, such as a three-phase separator, for the simultaneous separation of fat and residual solids from the protein solution.

In a further embodiment of the process or processes outlined above, the isoelectric precipitate is separated from the centrate in step e) by centrifugation or filtration optionally with a disc stack centrifuge.

In a further embodiment of the process or processes outlined above, the washed isoelectric precipitate is separated from the wash centrate in step f) by centrifugation or filtration optionally with a disc stack centrifuge.

In a further embodiment of the process or processes outlined above, the pH of the optionally washed isoelectric precipitate is raised in step g) using food grade sodium hydroxide, food grade potassium hydroxide or any other conventional food grade alkali and combinations thereof.

In a further embodiment of the process or processes outlined above, the optionally washed and optionally pH adjusted isoelectric precipitate is pasteurized prior to optional drying and wherein pasteurization optionally comprises heating the optionally washed and optionally pH adjusted isoelectric precipitate to a temperature of about 55° to about 85° C. for about 10 seconds to about 60 minutes, preferably about 60° C. to about 70° C. for about 10 minutes to about 60 minutes or about 70° C. to about 85° C. for about 10 seconds to about 60 seconds, and optionally the pasteurized optionally washed and optionally pH adjusted isoelectric precipitate is cooled, such as to a temperature of about 20° to about 35° C.

In a further embodiment of the process or processes outlined above, the optionally washed, optionally pH adjusted and optionally pasteurized isoelectric precipitate is subject to drying step h) by any conventional means such as spray drying or freeze drying to provide a sunflower protein product.

In a further embodiment of the process or processes outlined above, the optionally washed and optionally pH adjusted isoelectric precipitate may be jet cooked to a temperature of about 110 to about 150° C. for a time of about 10 seconds to about 1 minute, preferably about 140 to about 145° C. for about 40 to about 50 seconds, prior to drying step h).

In a further embodiment of the process or processes outlined above, the process or processes further comprise the steps of:
bi) optionally further processing the separated residual sunflower protein source obtained in step b), such as to recover residual protein; and
bii) optionally re-extracting the separated residual sunflower protein source obtained in step b) with fresh extraction solution to recover residual protein and separating the re-extraction protein solution from the residual sunflower protein source and optionally combining the re-extraction protein solution with the aqueous sunflower protein solution for further processing.

In a further embodiment, the present invention provides for a food or beverage comprising a sunflower protein product produced by the process or processes described herein, wherein optionally, the food or beverage is a fruit smoothie, a meat alternative, a dairy alternative product, or a baked good.

In a yet further embodiment, the present invention provides for a sunflower protein product prepared using isoelectric precipitation that has a L* value for the powder higher than 70 and/or has an a* value for the powder lower than 1.

### DETAILED DESCRIPTION

An initial step of the process of providing the sunflower protein products of the present invention involves solubilizing sunflower protein from a sunflower protein source derived from dehulled sunflower seeds (also known as a sunflower kernels). Dehulling prior to the protein extraction facilitates a better colour in the eventual purified protein product. The sunflower protein source may be derived from any variety of sunflower seed used for human food or animal feeding purposes. Confectionery (also known as non-oilseed), black oil (also known as oilseed) and conoil types of sunflower seed may be used. The sunflower protein source may be used in the full fat form, partially defatted form (e.g. cold pressed kernel cake/meal) or fully defatted form (e.g. pressed and solvent extracted kernel meal). Where the sunflower protein source contains an appreciable amount of fat, an oil removal step may be used during the process.

For the purpose of this disclosure, the terms “cake” and “meal” are used interchangeably. “Cake” is generally yielded from pressing and the solvent extraction of the cake yields a “meal”. Meal may also be considered a ground presscake. Pressed sunflower, encompassed by the terms “cake” and “meal” may be added to extraction solution without a grinding step when it is generally soft enough that it fragments when mixed with water or other appropriate solvent or liquid. The particle size of the sunflower protein source may vary but it is preferred that the sunflower protein source is in the form of granules or a powder to facilitate more rapid wetting and more thorough mixing with the extraction solution. As mentioned above, some sunflower protein sources, such as certain cakes/meals from cold pressing will fragment when mixed with extraction solution. The sunflower protein source may also be ground before the extraction step to achieve a desired particle size. Ground sunflower protein source may be referred to as a flour. The sunflower protein recovered from the sunflower protein source may be the protein naturally occurring in the sunflower seed or the proteinaccous material may be a protein modified by genetic manipulation but possessing characteristic hydrophobic and polar properties of the natural protein.

The sunflower protein products of the present invention may be prepared from sunflower protein source by either a batch process or a continuous process or a semi-continuous process. Protein solubilization from the sunflower protein source material is effected using an aqueous extraction solution comprising water, ascorbic acid or its derivative and an alkaline pH adjusting agent. The water used may be tap water or water having different levels of purity. Reverse osmosis (RO) purified water is preferred.

Ascorbic acid or its derivative is included in the extraction mixture to function as a reducing agent and/or anti-oxidant and inhibit the oxidation of sunflower phenolics such as chlorogenic acid into green or darkly coloured products. Suitable ascorbic acid derivatives include sodium ascorbate and calcium ascorbate. The quantity of ascorbic acid or its derivative employed may vary from about 0.01 to about 1 wt % of the solution, preferably about 0.05 to about 0.15 wt %, more preferably about 0.05 to about 0.10 wt %.

The alkaline pH adjusting agent is added to raise the pH of the extraction mixture to a value between the natural pH of the combination of the water, ascorbic acid or its derivative and the sunflower protein source and pH 8.5, or the pH of the extraction may be adjusted to a value between about 7.0 and about 8.0, preferably the pH is adjusted within the range of about 7.0 and about 7.5. Food grade sodium hydroxide, food grade potassium hydroxide or any other conventional food grade alkali and combinations thereof may be added to the extraction solution to adjust the pH of the extraction as needed. The food grade alkali is preferably added in aqueous solution form.

The solubilization of the protein is effected at a temperature of from about 1° to about 100° C. preferably about 15° to about 65° C., more preferably about 50° C. to about 60° C., preferably accompanied by agitation to decrease the solubilization time, which is usually about 1 to about 60 minutes, preferably about 10 to about 30 minutes. It is preferred to effect the solubilization to extract substantially as much protein from the sunflower protein source as is practicable, so as to provide an overall high product yield. The pH values of the extraction and subsequent steps typically refer to values measured at ambient temperature. For absence of doubt, when, for example, the extraction is conducted at an elevated temperature, the pH of the extraction mixture is such that a sample of extraction mixture cooled to ambient temperature has a pH reading in the specified range.

Extraction of the protein from the sunflower protein source, when conducted in a continuous operation, is carried out in any manner consistent with effecting a continuous extraction of protein from the sunflower protein source. In one embodiment, the sunflower protein source is continuously mixed with the extraction solution and the mixture is conveyed through a pipe or conduit having a length and at a flow rate for a residence time sufficient to effect the desired extraction in accordance with the parameters described herein.

The concentration of sunflower protein source in the extraction solution during the solubilization step may vary widely. Typical concentration values are about 5 to about 20% w/v, preferably about 5 to about 15% w/v.

It will be appreciated that reference to solubilizing encompasses both complete and partial solubilization of the protein from the sunflower protein source.

The protein extraction step has the additional effect of solubilizing fats which may be present in the sunflower protein source, which then results in the fats being present in the aqueous phase.

The aqueous phase resulting from the extraction step generally has a protein concentration of about 0.5 to about 5 wt %, preferably about 1 to about 5 wt %.

The aqueous phase resulting from the extraction step then may be separated from the residual sunflower protein source, in any conventional manner, such as by centrifugation and/or filtration to provide an aqueous sunflower protein solution. Preferably the aqueous phase resulting from the extraction step is separated from the bulk of the residual sunflower protein source using a decanter centrifuge. The resulting centrate may be further clarified using a disc stack centrifuge to remove finer solids. These finer solids may be combined with the residual solids collected from the decanter centrifugation and further processed as described below. The separation step may be conducted at the same temperature as the extraction step or at any temperature within the range of about 1° to about 100° C., preferably about 15° to about 65° C., more preferably about 50° to about 60° C. When the separation is done in more than one step (e.g. employing decanter centrifuge then disc stack centrifuge), different temperatures within the abovementioned range may employed for each step of the separation process. The separated residual sunflower protein source material (from decanter step alone or combined with the finer solids from the disc stack centrifugation) may be dried for disposal or further processed, such as to recover residual protein. Residual protein may be recovered by re-extracting the separated residual sunflower protein source with fresh extraction solution and the protein solution yielded upon clarification combined with the initial protein solution for further processing as described below. A counter-current extraction procedure may also be utilized. The separated residual sunflower protein source may alternatively be processed by any other conventional procedure to recover residual protein.

It will be appreciated that reference herein to a separation step and to separation of the aqueous phase and residual sunflower protein source is intended to refer to both complete separation as well as to at least partial separation. It is to be understood that trace or minor amounts of residual components may be found in the separated aqueous phase, for example but not limited to: residual protein source, finer solids, and/or fat/oil.

The aqueous sunflower protein solution may be treated with an anti-foamer, such as any suitable food-grade, non-silicone based anti-foamer, to reduce the volume of foam formed upon further processing. The quantity of anti-foamer employed is generally greater than about 0.0003% w/v. Alternatively, the anti-foamer in the quantity described may be added in the extraction step.

The separated aqueous sunflower protein solution may be subject to a defatting operation, if desired or required. Defatting of the separated aqueous sunflower protein solution may be achieved by any conventional procedure such as centrifugation and/or filtration. A three-phase centrifuge such as a three-phase separator may be used for the simultaneous separation of fat and residual solids from the protein solution with the three-phase centrifuge potentially being used instead of or in addition to the separation steps already described above. When a three-phase centrifuge is used in addition to the decanter and disc stack centrifuge described above, the order in which the disc stack and three-phase centrifuge steps are applied to the post-decanter protein solution may be varied. Solids collected by the three-phase centrifuge may be disposed of or further processed in combination with solids collected from the decanter centrifuge and disc stack centrifuge.

The aqueous sunflower protein solution may be treated with an adsorbent, such as granulated activated carbon, to remove colour and/or odour compounds. Such adsorbent treatment may be carried out under any conventional conditions, generally at the ambient temperature of the separated aqueous protein solution.

The aqueous sunflower protein solution is then adjusted in pH so that the pH is at or close to the isoelectric point of the majority of the sunflower protein. The pH adjustment results in the formation of an isoelectric precipitate. The pH of the aqueous sunflower protein is adjusted within the range of about pH 4.0 to about pH 5.0, preferably about pH 4.2 to about pH 4.8. The pH of the aqueous sunflower protein may be adjusted by the addition of any food grade acid such as food grade hydrochloric acid solution or food grade phosphoric acid solution. The isoelectric precipitation step may be conducted at any temperature within the range of about 1° to about 100° C., preferably about 15° to about 65° C., more preferably about 50° to about 60° C.

The isoelectric precipitate then may be separated from the residual aqueous sunflower protein solution, termed the centrate, in any conventional manner, such as by centrifugation and/or filtration. Preferably the isoelectric precipitate is separated from the centrate using a disc stack centrifuge. This separation step may be conducted at the same temperature as the isoelectric precipitation step or at any temperature within the range of about 1° to about 100° C., preferably about 15° to about 65° C., more preferably about 50° to about 60° C.

The isoelectric precipitate may then be optionally diluted with RO water then optionally dried to form a first sunflower protein product having a protein content of at least about 60 wt % (N×6.25) d.b., preferably about 65,70, 75, 80 or 85 wt % (N×6.25) d.b., more preferably about 90 wt % (N×6.25) d.b. Alternatively, the pH of the optionally diluted isoelectric precipitate may be raised to a value between about 6.0 and about 8.0, by any conventional means such as by the addition of food grade sodium hydroxide, food grade potassium hydroxide or any other conventional food grade alkali and combinations thereof prior to optional drying to form a sunflower protein product having a protein content of at least about 60 wt % (N×6.25) d.b., preferably about 65, 70, 75, 80 or 85 wt % (N×6.25) d.b., more preferably about 90 wt % (N×6.25) d.b. The food grade alkali is preferably added in aqueous solution form.

The isoelectric precipitate may be washed in order to remove contaminants and improve the purity and flavour of the product. The isoelectric precipitate may be washed by suspending the solids in between about 1 and about 20 volumes, preferably about 1 to about 10 volumes of water, preferably RO water with pH adjusting agent present so that the pH of the mixture is within the range of about 4.0 to about 5.0, preferably about 4.2 to about 4.8. The pH adjusting agent may be any food grade acid such as food grade hydrochloric acid solution or food grade phosphoric acid solution. The washing step may be conducted at any conventional temperature such as about 15° to about 65° C., preferably about 50° to about 60° C. The isoelectric precipitate is mixed with the wash solution for any conventional length of time, preferably 15 minutes or less. The washed isoelectric precipitate may then be separated from the wash solution by any conventional means such as by centrifugation using a disc stack centrifuge.

The separated wash solution, termed the wash centrate, may be added to the centrate from the initial separation of the isoelectric precipitate for further processing as discussed below. The washed isoelectric precipitate may be optionally diluted with water then optionally dried by any conventional means such as spray drying or freeze drying to provide a sunflower protein product having a protein content of at least about 60 wt % (N×6.25) d.b., preferably about 65, 70, 75, 80 or 85 wt % (N×6.25) d.b., more preferably about 90 wt % (N×6.25) d.b. Alternatively, the pH of the optionally diluted washed isoelectric precipitate may be adjusted to a value between about 6.0 and about 8.0, by any conventional means such as by the addition of food grade sodium hydroxide, food grade potassium hydroxide or any other conventional food grade alkali and combinations thereof, prior to optional drying. As a further alternative, the isoelectric precipitate may be pH adjusted during the washing step by adjusting the mixture of isoelectric precipitate and wash water to a pH between about 6.0 and about 8.0 using food grade alkali, then collecting the solids by centrifugation and optionally drying the solids. The food grade alkali is preferably added in aqueous solution form.

A pasteurization step may be employed on the optionally diluted and optionally pH adjusted isoelectric precipitate or optionally diluted and optionally pH adjusted washed isoelectric precipitate prior to the optional drying step. Such pasteurization may be effected under any conventional pasteurization conditions. Generally, the optionally diluted and optionally pH adjusted isoelectric precipitate or optionally diluted and optionally pH adjusted washed isoelectric precipitate are heated to a temperature of about 55° to about 85° C. for about 10 seconds to about 60 minutes, preferably about 60° C. to about 70° C. for about 10 minutes to about 60 minutes or about 70° C. to about 85° C. for about 10 seconds to about 60 seconds. The pasteurized optionally diluted and optionally pH adjusted isoelectric precipitate or optionally diluted and optionally pH adjusted washed isoelectric precipitate then may be cooled, such as to a temperature of about 20° to about 35° C.

As a further alternative, a jet cooking step may be employed on the optionally diluted and optionally pH adjusted isoelectric precipitate or optionally diluted and optionally pH adjusted washed isoelectric precipitate prior to the optional drying step. In such jet cooking, the optionally diluted and optionally pH adjusted isoelectric precipitate or optionally diluted and optionally pH adjusted washed isoelectric precipitate may be heated to a temperature of about 110 to about 150° C. for a time of about 10 seconds to about 1 minute. Preferably the product is heated to about 140° C. to 145° C. for about 40 to 50 seconds.

The isoelectric precipitate sunflower protein product has organoleptic and functional properties making it suitable for use in food and beverage products including but not limited to dairy alternatives (such as milk alternative beverages, frozen desserts, cheese alternatives and yogurt alternatives), meat alternatives (such as beef alternatives, pork alternatives and poultry alternatives), seafood alternatives (such as tuna alternatives, salmon alternatives and shrimp alternatives), grain products (such as pasta, bread and breakfast cereal), snacks or sweets (such as cookies, crackers, bar products, cakes, candy and chocolates), fats and oils products (such as margarines and dressings), condiments or sauces (such as tomato based sauces, non-tomato based sauces, dips and gravies), nutritional products (such as nutritional drinks and nutritional powders), sports drinks, energy drinks and smoothies. When prepared in lower pH form (pH of about 4 to about 5), the product is particularly well suited for lower pH food and beverage applications such as fruit smoothies. When prepared in neutral or near neutral pH form (pH of about 6 to about 8), the product is particularly well suited for food and beverage applications having a neutral or near neutral pH such as meat alternatives, dairy alternatives and baked goods. The sunflower protein product may be formulated into a food or beverage product to provide protein fortification. The sunflower protein product may be formulated into a food or beverage product to replace other protein ingredients or to replace non-protein functional ingredients. The isoelectric precipitate sunflower protein product may also be used in pet foods, animal feed, industrial products, cosmetic products and personal care products.

The centrate separated from the isoelectric precipitate may be combined with the wash centrate then concentrated and optionally diafiltered using membrane processing to provide a second sunflower protein product.

### EXAMPLES

### Comparative Example 1

This Example describes preparation of isoelectric precipitate product without the use of ascorbic acid.

30 kg of sunflower flour (partially defatted, made from kernels) was combined with 300 L of reverse osmosis purified (RO) water having a temperature of 63.9° C. and 25% NaOH solution to adjust the pH to 7.04 when measured at 42.5° C. Another 30 kg of sunflower flour (partially defatted, made from kernels) was combined with 300 L of reverse osmosis purified (RO) water having a temperature of 62.7° C. and 25% NaOH solution to adjust the pH to 7.19 when measured at 41.7° C. In total 0.92 kg of 25% NaOH solution was used to adjust the pH of the two batches. The mixtures were stirred for 15 minutes then a portion of the suspended solids (189.1 kg total) were removed by centrifugation using a decanter centrifuge to provide a protein solution having a protein content of 3.05 wt % and a pH of 7.20. The protein solution was then further clarified by centrifugation using a disc stack centrifuge to remove additional suspended solids (22.68 kg) and provide a protein solution having a protein content of 2.93 wt %. This protein solution was then fed to a three-phase separator which removed 41.86 kg of oil phase and another 16.64 kg of suspended solids and provided 350 L of protein solution having a protein content of 2.84 wt %. The pH of this solution was adjusted from 7.19 to 4.68 using 1 kg of HCl solution (concentrated HCl diluted with an equal volume of RO water), which resulted in isoelectric precipitation. A disc stack centrifuge was used to separate the isoelectric precipitate (44.96 kg, protein content of 16.11 wt %) from the centrate (330 L).

A 21.86 kg aliquot of isoelectric precipitate was adjusted in pH to 7.06 by the addition of 0.22 kg of 25% NaOH solution. The pH adjusted material was pasteurized at about 72° C. for 1 minute. The pasteurized material was spray dried to yield a product having a protein content of 89.62% (N×6.25) d.b. The product was termed SF11-L15-21A control SFIEP-01.

Another 23.08 kg aliquot of isoelectric precipitate was mixed with 90 L of 50° C. RO water and then centrifuged with a disc stack centrifuge. 18.72 kg of washed isoelectric precipitate was collected and the pH adjusted to 7.24 with 0.14 kg of 25% NaOH solution. The pH adjusted washed isoelectric precipitate was then pasteurized at about 72° C. for 2 minutes. The pasteurized material was spray dried to yield a product having a protein content of 93.66% (N×6.25) d.b. The product was termed SF11-L15-21A control SFIEP-02.

Note all cited pH values were from measurements conducted with the sample at room temperature unless otherwise noted.

### Example 1

30 kg of sunflower flour (partially defatted, made from kernels) was combined with 300 L of reverse osmosis purified (RO) water having a temperature of 62.1° C. 0.30 kg of ascorbic acid and 0.68 kg of 25% NaOH solution. The mixture was stirred for 10 minutes. A portion of the suspended solids (77.25 kg) were removed by centrifugation using a decanter centrifuge to provide a protein solution having a protein content of 3.21 wt % and a pH of 7.15. The protein solution was then further clarified by centrifugation using a disc stack centrifuge to remove additional suspended solids (6.8 kg) and provide a protein solution having a protein content of 2.72 wt %. This protein solution was then fed to a three-phase separator which removed 2.54 kg of oil phase and another 10.66 kg of suspended solids and provided 230 L of protein solution having a protein content of 2.42 wt %. The pH of this solution was adjusted from 7.12 to 4.77 using 0.63 kg of HCl solution, which resulted in isoelectric precipitation. A disc stack centrifuge was used to separate the isoelectric precipitate (11.72 kg, protein content of 32.40 wt %) from the centrate (225 L).

A 5.8 kg aliquot of isoelectric precipitate was diluted with 3 kg of RO water and then the pH of the diluted isoelectric precipitate raised to 6.98 by the addition of 80 g of 25% NaOH solution. The pH adjusted diluted isoelectric precipitate was then pasteurized at about 73° C. for 5 minutes. The pasteurized material was spray dried to yield a product having a protein content of 91.57% (N×6.25) d.b. The product was termed SF19-H16-22A SFIEP-01.

Another 5.74 kg aliquot of isoelectric precipitate was mixed with 22.96 kg RO water and then centrifuged in batches at 5,000 rpm for 5 minutes using a Sorvall RC3 lab centrifuge. 3.42 kg of washed isoelectric precipitate was collected, diluted with 2.93 kg of RO water and then the pH of the diluted washed isoelectric precipitate adjusted to 6.95 with 0.1 kg of 25% NaOH solution. The pH adjusted diluted washed isoelectric precipitate was then pasteurized at about 73° C. for 5 minutes. The pasteurized material was spray dried to yield a product having a protein content of 94.72% (N×6.25) d.b. The product was termed SF19-H16-22A SFIEP-02.

Note all cited pH values were from measurements conducted with the sample at room temperature unless otherwise noted. The extraction slurry was noted as having a pH of 7.10 at 45.3° C.

### Example 2

30 kg of sunflower flour (partially defatted, made from kernels) was combined with 300 L of reverse osmosis purified (RO) water having a temperature of 61.2° C., 0.30 kg of ascorbic acid and 0.68 kg of 25% NaOH solution. The mixture was stirred for an unrecorded length of time. A portion of the suspended solids (77.64 kg) were removed by centrifugation using a decanter centrifuge to provide a protein solution having a protein content of 3.36 wt % and a pH of 7.30. The pH of this solution was adjusted to 4.71 using HCl solution, which resulting in isoelectric precipitation. A disc stack centrifuge was used to separate the isoelectric precipitate (19.9 kg, protein content of 25.94 wt %) from the centrate (230 L). The collected isoelectric precipitate (19.9 kg) was combined with 59.7 kg RO water and the resulting slurry separated with a disc stack centrifuge. 17.88 kg of washed isoelectric precipitate and 55 L of wash centrate were collected. The 17.88 kg of washed isoelectric precipitate was diluted with 10 L of RO water and the pH of the diluted washed isoelectric precipitate raised to 7.26 by the addition of 0.26 kg of 25% NaOH solution. The pH adjusted diluted washed isoelectric precipitate was then pasteurized at about 73° C. for 5 minutes with 1 L of RO water added during heating. The pasteurized material was spray dried to yield a product having a protein content of 77.88% (N×6.25) d.b. The product was termed SF19-H17-22A SFIEP.

Note all cited pH values were from measurements conducted with the sample at room temperature unless otherwise noted. The extraction slurry was noted as having a pH of 7.10 at 53° C.

### Example 3

This Example contains an evaluation of the dry colour of the sunflower protein products prepared as described in Examples 1 and 2 as well as the product prepared in Comparative Example 1. Dry colour (CIEL*a*b*) was assessed using a HunterLab ColorQuest XE instrument operated in reflectance mode (RSEX) with an illuminant setting of D65 and an observer setting of 10°. The results are shown in the following Table 1.

TABLE 1
Dry colour of sunflower protein products

| Product | L* | a* | b* |
|---|---|---|---|
| SF11-L15-21A control SFIEP-01 | 63.12 | 1.23 | 16.78 |
| SF11-L15-21A control SFIEP-02 | 62.15 | 1.25 | 16.36 |
| SF19-H16-22A SFIEP-01 | 73.24 | 0.37 | 16.17 |
| SF19-H16-22A SFIEP-02 | 73.16 | 0.32 | 14.90 |
| SF19-H17-22A SFIEP | 70.40 | 0.77 | 14.85 |

As may be seen from the results of Table 1, products of the invention prepared using ascorbic acid had a higher L* value and so were lighter than the control products prepared without ascorbic acid. The products prepared with ascorbic acid also had less yellow colour (lower b*) and more particularly less red colour (lower a*) compared to the control products.

### Example 4

This Example contains an evaluation of the protein solubility of the sunflower protein products prepared as described in Examples 1 and 2 as well as the product prepared in Comparative Example 1.

A 100 ml beaker and magnetic stir bar were pre-weighed. Sufficient protein powder to supply 2 g of protein was weighed into the beaker. Approximately 10-15 mL of RO water was initially added and the sample stirred with the stir bar until the powder was thoroughly wetted. Once wetted, more water was slowly added to a total of approximately 40-45 mL and a timer was started. The sample was stirred for several minutes until the product was mostly dispersed. The pH of the sample was then adjusted to the target value with 0.5M NaOH or HCl as necessary. The sample was stirred on a magnetic stir plate set to a speed just below forming a vortex in the sample. The sample was stirred for a total of 60 minutes with the pH periodically checked and adjusted if necessary during this time. After the 60 minutes the pH of the sample was checked and corrected as necessary again and then additional RO water added to bring the sample weight to 50 g (protein concentration of 4% w/w) and mixed in. Approximately 20 ml of the dispersion was then transferred to a 50 ml centrifuge tube and centrifuged at 10,000 rpm (7,800 g) in a Sorvall SS-34 rotor for 10 minutes with the centrifuge set to 20° C. After the centrifugation was completed 10 ml of supernatant was removed from the centrifuge tube by pipet. Samples of the supernatant and the original dispersion were tested for protein content by combustion analysis (N×6.25).

Solubility (%)=(supernatant protein conc./original dispersion protein conc.)×100

The protein solubility of the sunflower protein products of Examples 1 and 2 and Comparative Example 1 is shown in Table 2.

TABLE 2
Solubility of sunflower protein products at different pH values

| | pH 4 | pH 5.5 | pH 7 |
|---|---|---|---|
| SF11-L15-21A control SFIEP-01 | 18.1 | 1.8 | 62.3 |
| SF11-L15-21A control SFIEP-02 | 33.1 | 1.3 | 61.0 |
| SF19-H16-22A SFIEP-01 | 22.2 | 4.3 | 69.0 |
| SF19-H16-22A SFIEP-02 | 30.8 | 4.4 | 69.1 |
| SF19-H17-22A SFIEP | 13.4 | 5.0 | 53.7 |

As may be seen from the results presented in Table 2, the ascorbic acid did not appear to have much effect on the solubility of the sunflower protein products

It will be appreciated that the examples and embodiments disclosed herein are intended to be non-limiting. They are illustrative of the invention and may be modified or altered. Such modifications and alterations within the concept and spirit of the intended invention.

1. A process for preparing a first sunflower protein product from a sunflower protein source, the sunflower protein product having a protein content of greater than 60, 65, 70, 75, 80, 85 or 90 wt % (N×6.25) d.b, the process comprising
a) extracting sunflower protein from an optionally partially defatted or optionally fully defatted, dehulled sunflower protein source, using an extraction solution comprising water containing an alkaline pH adjusting agent as well as ascorbic acid or a derivative thereof, to cause solubilization of the sunflower protein from the sunflower protein source to produce an aqueous phase and a residual sunflower protein source;
b) separating the aqueous phase from the residual sunflower protein source to produce an aqueous sunflower protein solution and a separated residual sunflower protein source;
c) optionally defatting the separated aqueous sunflower protein solution to at least partially remove fat from the separated aqueous sunflower protein solution;
d) adjusting the pH of the separated aqueous sunflower protein solution to an acidic value in the range of about pH 4.0 to about pH 5.0, preferably about pH 4.2 to about pH 4.8, to result in isoelectric precipitation of the sunflower protein;
e) separating the isoelectric precipitate from the remainder of the aqueous sunflower protein solution, termed the centrate;
f) optionally washing the isoelectric precipitate by mixing it with water with the pH of the system in the range of about 4.0 to about 5.0, preferably about pH 4.2 to about pH 4.8, and then separating the washed precipitate from the wash centrate;
g) optionally adjusting the pH of the optionally washed isoelectric precipitate to the range of about 6.0 to about 8.0, optionally about 6.5 to about 7.5, and optionally the isoelectric precipitate is in a form suited for use in neutral or near-neutral food applications;
h) optionally drying the optionally washed and optionally pH adjusted isoelectric precipitate;
wherein step a) is optionally carried out using a counter-current extraction procedure;
wherein the extraction is optionally carried out in a continuous operation or a batch operation; and/or
wherein step g) is optionally not performed and the isoelectric precipitate is in a form suited for use in low pH food and beverage applications.

2. The process of claim 1, wherein the centrate formed in step e) may be combined with the wash centrate formed in step f) and concentrated and optionally diafiltered using membrane processing to provide a second sunflower protein product.

3. The process of claim 1, wherein the dehulled sunflower protein source in a) is derived from confectionery (also known as non-oilseed), black oil (also known as oilseed) or conoil type sunflower seed.

4. The process of claim 1, wherein the extraction solution contains ascorbic acid or a derivative thereof in an amount of from about 0.01 to about 1 wt % of the solution, preferably about 0.05 to about 0.15 wt %, more preferably about 0.05 to about 0.10 wt %.

5. The process of claim 1, wherein the pH of the extraction is adjusted up to any value between the natural pH and about 8.5, or the pH may be adjusted within the range of about 7.0 to about 8.0, or the pH may be adjusted within the range of about 7.0 to about 7.5, wherein the alkaline pH adjusting agent in the extraction solution is food grade sodium hydroxide, food grade potassium hydroxide or any other conventional food grade alkali and combinations thereof.

6. The process of claim 1, wherein solubilization of the protein in step a) is effected at a temperature of from about 1° to about 100° C., preferably about 15° to about 65° C., more preferably about 50° C. to about 60° C., preferably accompanied by agitation, for a time of about 1 to about 60 minutes, preferably about 10 to about 30 minutes.

7. The process of claim 1, wherein the concentration of the sunflower protein source in the extraction solution during the extraction step is selected from the group consisting of about 5 to about 20% w/v and about 5 to about 15% w/v.

8. The process of claim 1, wherein the aqueous phase resulting from the extraction step generally has a protein concentration of about 0.5 to about 5 wt %, preferably about 1 to about 5 wt %.

9. The process of claim 1, wherein separation in step (b) comprises centrifugation and/or filtration optionally with a decanter centrifuge and a disc stack centrifuge and is conducted at the same temperature as the extraction step or at any temperature within the range of about 1° to about 100° C., preferably about 15° to about 65° C., more preferably about 50° to about 60° C.

10. The process of claim 1, wherein the process further comprises treating the aqueous sunflower protein solution with an anti-foamer, such as any suitable food-grade, non-silicone based anti-foamer, to reduce the volume of foam formed upon further processing, wherein the quantity of anti-foamer employed is generally greater than about 0.0003% w/v, and wherein the anti-foamer is optionally added during the extraction step a).

11. The process of claim 1, wherein defatting of the separated aqueous sunflower protein solution in step c) is achieved by centrifugation and/or filtration and/or the use of a three-phase centrifuge, such as a three-phase separator, for the simultaneous separation of fat and residual solids from the protein solution.

12. The process of claim 1, wherein the isoelectric precipitate is separated from the centrate in step e) by centrifugation or filtration optionally with a disc stack centrifuge.

13. The process of claim 1, wherein the washed isoelectric precipitate is separated from the wash centrate in step f) by centrifugation or filtration optionally with a disc stack centrifuge.

14. The process of claim 1, wherein the pH of the optionally washed isoelectric precipitate is raised in step g) using food grade sodium hydroxide, food grade potassium hydroxide or any other conventional food grade alkali and combinations thereof.

15. The process of claim 1, wherein the optionally washed and optionally pH adjusted isoelectric precipitate is pasteurized prior to optional drying and wherein pasteurization optionally comprises heating the optionally washed and optionally pH adjusted isoelectric precipitate to a temperature of about 55° to about 85° C. for about 10 seconds to about 60 minutes, preferably about 60° C. to about 70° C. for about 10 minutes to about 60 minutes or about 70° C. to about 85° C. for about 10 seconds to about 60 seconds, and optionally the pasteurized optionally washed and optionally pH adjusted isoelectric precipitate is cooled, such as to a temperature of about 20° to about 35° C.

16. The process of claim 1, wherein the optionally washed, optionally pH adjusted and optionally pasteurized isoelectric precipitate is subject to drying step h) by any conventional means such as spray drying or freeze drying to provide a sunflower protein product.

17. The process of claim 1, wherein the optionally washed and optionally pH adjusted isoelectric precipitate may be jet cooked to a temperature of about 110 to about 150° C. for a time of about 10 seconds to about 1 minute, preferably about 140 to about 145° C. for about 40 to about 50 seconds, prior to drying step h).

18. The process of claim 1, further comprising the steps of:
bi) optionally further processing the separated residual sunflower protein source obtained in step b), such as to recover residual protein; and
bii) optionally re-extracting the separated residual sunflower protein source obtained in step b) with fresh extraction solution to recover residual protein and separating the re-extraction protein solution from the residual sunflower protein source and optionally combining the re-extraction protein solution with the aqueous sunflower protein solution for further processing.

19. A food or beverage comprising a sunflower protein product produced by the process of claim 1, wherein optionally, the food or beverage is a fruit smoothie, a meat alternative, a dairy alternative product, or a baked good.

20. A sunflower protein product prepared using isoelectric precipitation that has a L* value for the powder higher than 70 and/or has an a* value for the powder lower than 1.
